(19) United States
(12) Patent Application Publication
(10) Pub. No.: US 2005/0287168 A1
(43) Pub. Date: Dec. 29, 2005

(54) Recombinant spores

(76) Inventor: Simon Michael Cutting, Egham, Surrey (GB)

Correspondence Address: THE MAXHAM FIRM, 750 "B" STREET, SUITE 3100, SAN DIEGO, CA, 92101, US

(21) Appl. No.: 10/506,749

(22) PCT Filed: Mar. 7, 2003
(86) PCT No.: PCT/GB03/00966

§ 371 Date: Jun. 27, 2005

(30) Foreign Application Priority Data
Mar. 7, 2002 (GB) 0205376.7

(51) Int. Cl. A61K 39/02; C12N 1/21

(52) U.S. Cl. 424/200.1; 435/252.31; 435/252.3

(57) ABSTRACT

A spore which is genetically modified with genetic code comprising at least one genetic construct encoding a therapeutically active compound and a targeting sequence or a vegetative cell protein.

### BACKGROUND OF THE INVENTION

1. Field of the Invention

This invention relates to the germination of spores and in particular, but not exclusively, to spores of Bacillus species of bacteria and uses thereof.

2. Discussion of Related Art

Infection is the leading cause of death in human populations. The two most important contributions to public health in the past 100 years have been sanitation and vaccination, which together have dramatically reduced deaths from infectious disease.

The development of improved vaccination strategies has always been of the utmost importance for a number of reasons.

Firstly, to provide better levels of immunity against pathogens which enter the body primarily through the mucosal surfaces. Vaccines are generally given parenterally. However, many diseases use the gastrointestinal (GI) tract as the primary portal of entry. Thus, cholera and typhoid are caused by ingestion of the pathogens Salmonella typhi and Vibrio cholera and subsequent colonisation at (V. cholera) or translocation (S. typhi) across the mucosal epithelium (lining the GI tract). Similarly, TB is initially caused by infection of the lungs by Mycobacterium tuberculosis. Immunisation via an injection generates a serum response (humoral immunity) which includes a predominant IgG response which is least effective in preventing infection. This is one reason why many vaccines are partially effective or give short protection times.

Secondly, to provide needle-less routes of administration. A major problem of current vaccination programmes is that they require at least one injection. For example tetanus vaccine. Although protection lasts for 10 years children are initially given three doses by injection and this should be followed by a booster every 5 years. In developed countries many people will choose not to take boosters because of ‘fear of injection’. In contrast, in developing countries where mortality from tetanus is high the problems lie with using needles that are re-used or are not sterile.

Thirdly, to offer improved safety and the minimisation of adverse side effects. Many vaccines consist of either live organisms which are either rendered non-pathogenic (attenuated) or are inactivated in some way. While in principle, this is considered safe there is evidence showing that safer methods must be developed. For example, in 1949 (the Kyoto incident) 68 children died from receiving a contaminated diphtheria vaccine (Health 1996). Likewise, in the Cutter incident of 1995 105 children developed polio. It was found that the polio vaccine had not been correctly inactivated with formalin. Many other vaccines, for example the MMR (measles-mumps-rubella) vaccine and the whooping cough vaccine (Health, 1996) are plagued with rumours of side effects.

Fourthly, to provide economic vaccines for developing countries where poor storage and transportation facilities prevent effective immunisation programmes. In developing countries where a vaccine must be imported it is assumed that the vaccine will be stored and distributed correctly. The associated costs of maintaining vaccines in proper hygienic conditions under refrigeration are significant for a developing country. For some vaccines such as the oral polio vaccine and BCG vaccine the vaccines will only survive for one year at 2-8° C. (Health, 1996). The need for a robust vaccine that can be stored indefinitely at ambient temperature is a high priority now for developing countries. This type of vaccine should ideally be heat stable, able to withstand great variations in temperature as well as desiccation. Finally, a vaccine that is simple to produce would offer enormous advantages to a developing country and would potentially be producable in that country.

It is an aim of the present invention to provide a spore in which said spore may be genetically modified to produce a medicament upon germination into a vegetative cell.

### SUMMARY OF THE INVENTION

Accordingly, the present invention, provides a spore which is genetically modified with genetic code comprising at least one genetic construct encoding a therapeutically active compound and a targeting sequence or a vegetative cell protein.

It is an advantage of the present invention in that the use of spores to administer vaccines will eliminate the need for injections and the problems associated with needles in developing countries. In addition to this, spores are stable and are resistant to heat and desiccation, therefore overcoming problems of storing vaccines in developing countries. Spores are easy to produce, and can be done at low cost making the production of vaccines in accordance with the invention economical and finally, as a non-pathogen and its current use as an oral probiotic, the use of Bacillus subtilis makes this a safer vaccine system than those currently available.

It is a further advantage of the invention that the spores elicit an immune response at the mucosal membranes. This makes the vaccination more effective against mucosal pathogens e.g. S. typhi, V. cholera and M. tuberculosis.

A vaccine delivered at the mucosal surfaces will be more effective in combating those diseases which infect via the mucosal route. The mucosal routes of vaccine administration would include oral, intra-nasal and/or rectal routes.

It is a further advantage of the present invention in that when said spore is administered to an animal, said spore germinates into a vegetative cell, said vegetative cell expresses said chimeric gene, wherein said chimeric gene comprises said medicament and said protein in order to elicit an immune response against said antigen.

It is yet a further advantage of the present invention that mucosal immunity can be achieved using B. subtilis cells. It had been assumed that B. subtilis cells would have to be engineered to enhance their ability to interact with phagocytic cells (macrophages/dendritic cells) of the mucosa. This assumption is based upon the fact that some vaccine systems using heterologous antigen presentation use colonising bacteria (such as Lactobacilli or Streptococci) for antigen delivery. U.S. Pat. No. 5,800,821 has specifically stated the need to express the Yersinia pestis invasion protein (Inv) in B. subtilis cells to promote interaction with the mucosa. Our present invention has shown this assumption to be unfounded and unnecessary.

Preferably the therapeutically active compound is an antigen or a medicament or a precursor to an antigen or a medicament. Preferably the gene construct is a chimeric gene. Preferably the spore is of Bacillus or Clostridia.

The genetic modification is accomplished by transformation of a mother cell using a vector containing the chimeric gene, using standard methods known to persons skilled in the art and then inducing the mother cell to produce spores according to the invention.

The gene construct may be under the control of one or more of, each or independently, an inducible promoter, a promoter or a strong promoter or modified promoter. The gene construct may have one or more enhancer elements or upstream activator sequences and the like associated with it.

The gene construct may comprise an inducible expression system. The inducible expression system is such that when said spore germinates into a vegetative cell the therapeutically active compound is not expressed unless exposed to an external stimulus e.g. pH or a pharmaceutical.

Generally the spore germinates in the intestinal tract. More preferably the spore germinates in the duodenum and/or the jejunum of the intestinal tract.

The genetic code may comprise DNA and/or cDNA. It will be appreciated that the term genetic code is intended to embrace the degeneracy of codon usage.

It has surprisingly been found not to be necessary to prime the spores to germinate prior to oral administration. This is particularly true of spores of the Bacillus species.

The spores are not heat inactivated prior to administration.

The vegetative cell only expresses a chimeric gene product after germinating from a spore. This may be achieved for example by, making a genetic construct of the antigen with a genetic construct of a protein expressed only in the vegetative state (e.g. the membrane associated protein OppA). This protein is not a spore coat protein.

The antigen is preferably at least a fragment of tetanus toxin fragment C or labile toxin B sub unit.

This aspect of the invention enables the antigen to be exposed to the human or animal body such that said antigen can elicit an immune response.

The antigen is preferably an antigen which, in use, is adapted to elicit an immune response.

The protein used may be any that are expressed only in the vegetative state. The protein may be a protein that is expressed in the cell barrier.

When we say a protein that is expressed in the cell barrier, we mean any protein (including lipoproteins and glycoproteins) that are expressed in, or in association with, the cell membrane, either intra-cellularly or extra-cellularly of the same; a protein expressed integrally with the cell membrane, a protein associated with the cell wall, either within the periplasmic space or externally of the cell wall or a protein expressed integrally of the cell wall.

This aspect enables a spore to be given orally to deliver the antigen. Alternatively, the spore may be administered via an intra-nasal or rectal route.

The antigen may be a chimera with different vegetative cell proteins. By having the genetic construct encoding the antigen with a genetic construct encoding one or more different vegetative cell proteins it may be possible to provide a temporal expression of the antigen. For example, the medicament may be expressed as a chimera with a vegetative cell protein that is expressed all the time, e.g. OppA or rrnO, therefore providing a constant “dose” of antigen.

Alternatively, the genetic construct encoding the antigen may be with a genetic construct encoding a vegetative cell protein that is expressed intermittently and therefore upon expression of the chimera said chimera is capable of administering the medicament in a time-controlled manner. The genetic construct encoding the medicament may also be with a genetic construct of a vegetative cell protein that is expressed initially at a high concentration but which then decreases over time, thus upon expression, the chimera is capable of administering an initial high dose of the antigen.

The temporal administration of doses could be customised by using, for example, one or more of the above genetic constructs.

Alternatively, the genetic construct encoding the antigen may be with a genetic construct encoding a soluble cytoplasmic vegetative cell protein, e.g. rrnO.

When the antigen is expressed as a chimera with a soluble cytoplasmic protein, said soluble cytoplasmic protein may function to target the whole chimera to the periplasmic space for subsequent secretion by a passive mechanism, (e.g. diffusion). Alternatively, the soluble protein may target the chimera for secretion by an active mechanism, for example, by Type I, Type II or Type III secretion.

The genetic construct of the soluble cytoplasmic protein may wholly or partially comprise a signal sequence.

According to a second aspect, the present invention provides a spore which is genetically modified with genetic code comprising a genetic construct encoding an antigen and a signal sequence, wherein said signal sequence is adapted to target said antigen to a specific part of the vegetative cell. For example, the signal sequence may direct the medicament for secretion, for example active secretion (Type I, Type II or Type III secretion), or for post-translational processing by the vegetative cell, e.g. glycosylation.

The vegetative cells may lyse in the intestinal tract and subsequently release the antigen as a chimera.

When the antigen is expressed with a vegetative cell-barrier protein the antigen may generally elicit a localised immune response by the immune system in the immediate vicinity of the vegetative cell. Alternatively, when the antigen is expressed in the cytoplasm and the vegetative cells subsequently lyse and release the antigen or the antigen is secreted by the vegetative cells said antigen may generally elicit a diffuse immune response over a larger area than the immediate vicinity of the vegetative cell.

The spore, according to the present invention, may be genetically engineered to comprise one or more enzymes capable of transforming biological precursors, such that upon germination said one or more enzymes are expressed and synthesise one or more antigens by transformation of said biological precursors. For example by
a) processing a biological precursor, e.g. a hormone. The hormone may be a chimeric protein expressed in the vegetative cell e.g. a cell-barrier protein, which requires subsequent processing (i.e. release from the cell via an enzyme cleavage site) to be activated, or
b) the biosynthesis, or processing, of a non-protein compound, e.g. steroid hormones and painkillers synthesised from available biological precursor materials, or processing of a pro-drug into an active drug.

According to a further aspect, the present invention provides according to the invention in which said spore is genetically modified with genetic code comprising at least one genetic construct encoding a medicament and a vegetative cell protein, as a chimeric gene.

The medicament may be one or more of:
a) Proteins, including enzymes, antigens, antibodies, hormones or metabolic precursors;
b) Vaccines;
c) Endorphins and the like.

According to a further aspect, the present invention provides spores according to the invention for use in treatment of a medical condition.

According to a further aspect, the present invention provides a composition comprising at least two different spores according to the invention and, optionally, a pharmaceutically acceptable excipient, in which said at least two different spores express at least two different antigens or medicaments, especially for use in treatment of a medical condition.

According to a further aspect, the present invention provides use of a spore according to the invention in the manufacture of a medicament for the treatment of a medical condition.

According to a third aspect, the present invention provides a composition comprising a spore according to the invention in association with a pharmaceutically acceptable excipient or carrier.

Suitable pharmaceutically acceptable carriers would be well known to a person of skill in the art.

According to a further aspect, the present invention provides a composition according to the invention for use in a method of medical treatment.

The invention also provides use of the composition according to the invention in the manufacture of the medicament for use in the treatment of a medical condition.

A method of medical treatment would comprise treating a medical condition e.g. a disease or administering a vaccine. Medical conditions for treatment by the invention include, for example, inflammation, pain, hormonal imbalances and/or intestinal disorders.

According to a further aspect, the present invention provides a method of medical treatment, which method comprises the steps of
a) Orally administering a spore according to the invention to a person or animal in need of medical treatment;
b) Said spore germinating into a vegetative cell in the intestinal tract;
c) Said vegetative cell expressing a therapeutically active compound for use in the medical treatment.

### BRIEF DESCRIPTION OF THE DRAWING

The invention will now be described merely by way of example, with reference to the accompanying figures, of which:

FIG. 1 shows a map of the pDG364 cloning vector showing the multiple cloning site, catgene and front and rear portions of the amyE gene. Restriction sites that can be used for linearisation are indicated; nucleotide positions are noted in brackets.

FIG. 2 illustrates the double-crossover recombinational event that generates a partial diploid using the cloning vector pDG364.

FIG. 3a shows Western blotting of size fractionated (12% SDS-PAGE) proteins. A polyclonal antiserum to TTFC was used. Lane 1, non-recombinant strain PY79 vegetative cells, Lane 2, strain PY79 carrying amyE::oppA-TTFC. Lane 3, purified TTFC protein.

FIG. 3b shows Western blotting of size fractionated (12% SDS-PAGE) proteins extracted from either the spore surface of non-recombinant PY79 spores (Lane 1), spores expresssing CotA::LTB (lane 2) and purified LTB protein. [note: The strain used for Lane 2 had the genotype amyE::oppA-TTFC thrC::cotA-LTB]

FIG. 3c shows Western blotting using a polyclonal anti-TTFC serum to size fractionated proteins from sonicated extracts of vegetative cells. Lane 1, non-recombinant PY79 cells. Lane 2, amyE::oppA-TTFC thrC::cotA-LTB cells and Lane 3; purified TTFC protein.

FIG. 4 shows anti-TTFC serum IgG titers following intraperitoneal immunisation with recombinant B. subtilis vegetative cells. Individual samples from groups of eight mice immunised intraperitoneally ( ) with 1×10⁹ wild-type (●) or OppA-TFFC expressing B. subtilis cells (Δ) were tested by ELISA for TTFC-specific IgG. Sera from a naïve control group (◯) were also assayed. The end-point titer was calculated as the dilution of serum producing the same optical density as a 1/40 dilution of a pooled preimmune serum.

FIG. 5 shows anti-TTFC serum IgG titers following oral immunisation with recombinant B. subtilis spores. Individual samples from groups of eight mice immunised orally ( ) with 1.7×10¹⁰ wild-type (●) or OppA-TTFC recombinant B. subtilis spores (Δ) were tested by ELISA for TTFC-specific IgG. Sera from a naïve control group (◯) were also assayed. The end-point titer was calculated as the dilution of serum producing the same optical density as a 1/40 dilution of a pooled preimmune serum.

FIG. 6 shows anti-TTFC serum IgG titers following oral immunisation with recombinant B. subtilis spores. Individual samples from groups of eight mice immunised orally ( ) with 1.7×10¹⁰ wild-type (●) or OppA-TTFC CotA-LTB recombinant B. subtilis spores (Δ) were tested by ELISA for TTFC-specific IgG. Sera from a naïve control group (◯) were also assayed. The end-point titer was calculated as the dilution of serum producing the same optical density as a 1/40 dilution of a pooled preimmune serum.

FIG. 7 shows anti-LTB serum IgG titers following oral immunisation with recombinant B. subtilis spores. Individual samples from groups of eight mice immunised orally ( ) with 1.7×10¹⁰ wild-type (●) or OppA-TTFC CotA-LTB recombinant B. subtilis spores (Δ) were tested by ELISA for TTFC-specific IgG. Sera from a naïve control group (●) were also assayed. The end-point titer was calculated as the dilution of serum producing the same optical density as a 1/40 dilution of a pooled preimmune serum.

FIG. 8 shows Survival of vegetative cells vs spores in GIT of a mouse model. Groups of inbred BALB/C mice were orally dosed with vegetative cells or spores of B. subtilis strain SC2362. Faecal and intestinal samples were assessed for total viable counts at indicated time points. Panel A, oral dose of 2.4×10¹⁰ vegetative cells; Panel B, oral dose of 2.1×10⁸ spores. Data were presented as arithmetic means and error bars were standard deviations.

FIG. 9 shows survival of vegetative cells and spores in simulated gastric condition. Vegetative cells of B. subtilis, E. coli, C. rodentium, and spores of B. subtilis (Panels A to D respectively) were treated (●) in simulated gastric conditions, and viability was assessed at indicated time points in comparison with untreated (∘) samples. Percentages were counts compared to original inocula. Data were presented as arithmetic means of duplicate independent experiments.

FIG. 10 shows survival of vegetative cells and spores in simulated intestinal condition. Vegetative cells of B. subtilis, E. coli, C. rodentium, and spores of B. subtilis (Panels A to D respectively) were treated (●) in simulated intestinal condition, and viability was assessed at indicated time points in comparison with untreated (∘) samples. Percentages were counts compared to original inocula. Data were presented as arithmetic means of duplicate independent experiments.

FIG. 11 shows spore germination in simulated intestinal condition. Spore suspensions of B. subtilis strain PY79 were examined for germination in AGK solution with (●) or without (∘) the presence of bile salts. OD600 nm readings were taken at indicated time points following the addition of L-alanine to trigger germination. Percentages were of OD readings compared to original suspensions. Data are presented as arithmetic means of duplicate independent experiments.

FIG. 12 shows expression and quantification of expressed b-galactosidase. Panel A: Samples of PY79 and SC2362 (rrnO-lacZ) grown in LB were labelled with mouse anti-β-galactosidase antibody followed by anti-mouse IgG-TRITC conjugate (red fluorescein). Panel B: Coomassie stained 10% SDS-PAGE (upper panel) and β-galactosidase-specific Western blot (lower panel) profiles of fractionated cell extracts from PY79 (spo+), SC2362 (rrnO-lacZ) and DL169 (gerD-cwlBD D::neo rrnO-lacZ). Arrows indicate β-galactosidase at the predicted mwt. of 117 kDa. Panel C: Dot blot experiments performed with the indicated concentrations of β-galactosidase (in mg) in cell extracts from strains PY79 (spo+), SC2362 (rrnO-lacZ) and DL169 (gerD-cwlBD D::neo rrnO-lacZ). Purified β-galactosidase dilutions (in ng) are spotted on the left (lane +). Anti-β-galactosidase primary antibodies and secondary antirabbit peroxidase-conjugated antibodies were used. Reactions were visualized by ECL as described in the Material and Methods section of Example 2.

FIG. 13 shows systemic responses after oral delivery of spores carrying rrnO-lacZ gene. Groups of inbred BALB/C mice were orally dosed (↑) with 2×10¹⁰ spores/dose or 3×10¹⁰ vegetative cells/dose of B. subtilis. Individual serum samples were tested by ELISA for anti-β-galactosidase specific IgG. Sera from a naïve, non-immunised, control group (∘) were also included as well as mice dosed with PY79 spores (⋄), PY79 vegetative cells (u), SC2362 spores (l), SC2362 vegetative cells (n), DL169 spores (Δ), and DL169 vegetative cells (black triangle). Data were presented as arithmetic means and error bars were standard deviations.

FIG. 14 shows analysis of anti-β-galactosidase IgG subclasses. Groups of inbred BALB/C mice were orally dosed (−) with 2×10¹⁰ spores/dose of B. subtilis strain SC2362 (Panel A), or 3×10¹⁰ vegetative cells/dose of strain SC2362 (Panel B), or vegetative cells of strain DL169 (Panel C). Individual serum samples were tested by ELISA for anti-β-galactosidase specific IgG1 (∘), IgG2a(Δ), and IgG2b (□) subclasses. Data were presented as arithmetic means and error bars were standard deviations.

### DETAILED DESCRIPTION OF THE PREFERRED EMBODIMENTS

The invention will now be illustrated with reference to the following non-limiting Examples.

### EXAMPLE 1

Construction of Recombinant Genes

In the following Example, the oppA gene has been used for recombinant gene constuction. This gene is well studied and forms part of an operon. OppA acts as the receptor for the initial uptake of peptides by the oligopeptide permease (Opp). The OppA protein in B. subtilis is well expressed and is involved in competence as well as spore formation (it is referred to as SpoOK).

In the cytoplasmic membrane, fusion of a gene sequence to the 3′-end of oppA would allow expression of a recombinant protein (ProteinX) where OppA-Protein X is assembled into the membrane with the C-terminal domain (carrying the fused domain) exposed to the outer face of the membrane. In a Gram-positive this means that the antigen would be exposed to the space between the membrane and peptidoglycan wall.

Since oppA is involved in spore formation, any modification made to this protein must be made in trans to an intact copy. That is, one copy of oppA must be held intact on the chromosome. To achieve this, we use the amyE loci (encoding amylase) to carry chimeric genes. Thus, a recombinant oppA-genX chimera is placed at the amyE locus in cells carrying an intact oppA gene (and opp locus) at the normal chromosomal position. An alternative locus is thrC for which cloning vectors are available.

In the preferred embodiment of this invention, the Gram positive bacterium Bacillus subtilis is used. The excellent genetics associated with this organism and the intense study of its genome make it, after Escherichia coli, the second most studied prokaryote. This organism is regarded as a non-pathogen and is classified as a novel food which is currently being used as a probiotic for both human and animal consumption. The single, distinguishing feature, of this microorganism is that it produces an endospore as part of its developmental life cycle when starved of nutrients. The mature spore, when released from its mother cell can survive in a metabolically dormant form for hundreds, if not thousands of years.

a) Construction of Gene Chimeras

i) amyE::oppA-TTFC. TTFC (tetanus toxin fragment C) is a 47 kDa component of tetanus toxin produced by Clostridium tetani. TTFC was fused to oppA and introduced at the amyE locus.

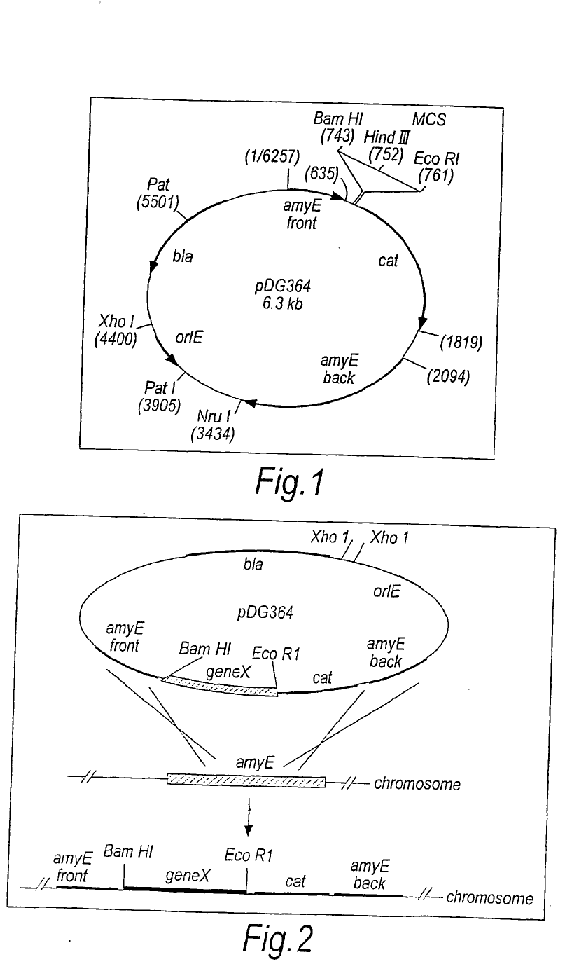

PCR was used to amplify i) appropriate sequences of the tetC gene (carried in vector pTet8) encoding the 47 kDa TTFC fragment, ii) the 5′-region of the oppA gene including its promoter. The oppA and tetC PCR products were fused using restriction digestion and ligation of 3′ and 5′ ends (using embedded cleavage sites in the PCR primers). The oppA-TTFC fragment was then cloned into the pDG364 vector (FIG. 1) at the multiple cloning sites.

FIG. 1 shows the plasmid pDG364 and this vector has been described elsewhere. The essential features of this vector are the right and left flanking arms of the amyE gene (referred to as amyE front and amyE back). Cloned DNA (ie, the cot-Antigen chimera) is introduced into the multiple cloning sites using general PCR techniques. The clone validated and the plasmid clone linearised by digestion with enzymes recognising the backbone sequences (eg, PstI). The linearised DNA is now used to transform competent cells of B. subtilis using selection for the antibiotic resistance carried by the plasmid (chloramphenicol resistance). As shown in FIG. 2, the linearised plasmid will only integrate via a double crossover recombination event using the front and back flanking arms of amyE for recombination. In the process the cloned DNA is introduced into the amyE gene and the amyE gene inactivated in the process. This procedure minimises damage to the chromosome and does not impair cell growth, metabolism nor spore formation.

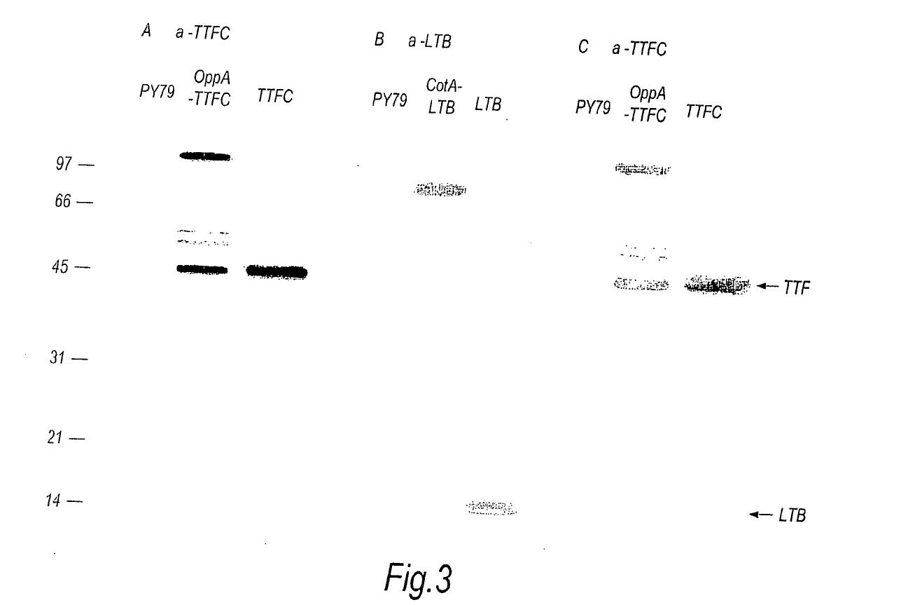

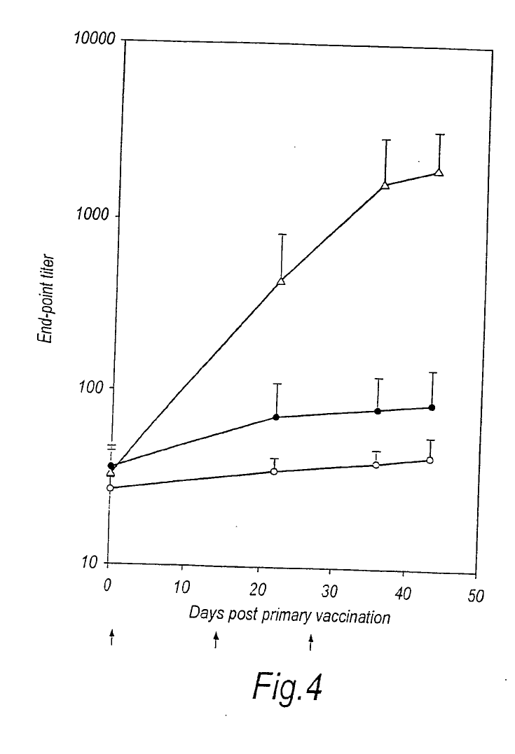

The clone was verified by DNA sequencing across junctions and the vector linearised and then introduced into the chromosome of B. subtilis using a double crossover recombination (FIG. 2). Selection for Chloramphenic-resistant and screening for amylase-negative colonies ensured a double crossover as shown in FIG. 2 and is described elsewhere. Cells carrying this construct at amyE were tested for the presence of TTFC by Western blotting as shown in FIG. 3 using a polyclonal antiserum to TTFC.
ii) amyE::oppA-TTFC thrC::cotAO-LTB. This construct carried two constructions placed at the amyE and thrC loci.

In this construction, we used a plasmid carrying a chimeric gene fusion of the cotA gene fused to the Escherichia coli 11 kDa Labile toxin Fragment B (LTB). PCR technology was used to amplify LTB and cotA sequences and fuse these together, in frame. CotA encodes a major protein 65 kDa from the spore coat surface layers. In the first step, the cotA-LTB chimera was constructed using the vector pDG1664. pDG1664 is similar to pDG364 (FIG. 1) but carries the erythromycin-resistance gene (erm). Thus, selection for a double crossover recombination event is made by selection for Erm^{R}. The second important feature of pDG1664 is that insertion uses the front and back (left and right) arms of the thrC locus enabling insertion and disruption of the thrC locus. Using this strategy, we made thrC::cotA-LTB cells, induced these to sporulate and then examined the spore coat proteins for the presence of CotA-LTB using a mouse polyclonal serum to LTB (FIG. 3). Having demonstrated adequate expression of the CotA-LTB chimera on the spore surface we used chromosomal DNA of thrC::cotA-LTB to transform competent cells of a strain carrying amyE::oppA-TTFC. Selection was made for Erm^{R} and the transformants would carry two chimeric genes, oppA-TTFC and cotA-LTB. The presence of both chimeras was confirmed by Western blotting of vegetative cells with anti-TTFC serum and for spore coats proteins with ant-LTB serum.

b) Multiple Antigen Presentation

To achieve multiple antigen presentation on the spore coat, it is necessary to use either pDG364 and pDG1664 plasmid vectors. One chimeric gene is made in pDG364 and the chimera introduced at the amyE locus and a second chimera made in pDG1664 and introduced at the thrC locus. This can be achieved since each transformational event requires a separate drug-resistant selection.

We have used this approach to express LTB on the spore surface and TTFC within the vegetative cells. This feature is attractive and could be used for bivalent vaccinations. Alternatively we could use TTFC expression on the spore (fused to CotA) and also from the vegetative cells (fused to OppA) enabling even higher doses.

c) Strain Validation

In our approach, we do not reason that it is necessary to determine that the chimeric gene product is surface displayed, ie, on the surface-most layers of the cell. (This could be achieved using FACS analysis or some other type of flow cytometry or using immunoflorscence). Our approach assumes that interaction of the vegetative cells with the mucosa must be achieved and in doing so, so long as an antigen is on or near the surface it will be able to stimulate immunity. This may well include cell-mediated immunity deriving from phagocytosis of the spore by macrophages or dendritic cells. In our rationale, it may actually be beneficial for the antigen to be partially protected within the cell envelope. Demonstration of immunogencity via mucosal immunisation is sufficient for further development.

d) Parenteral Immunisation

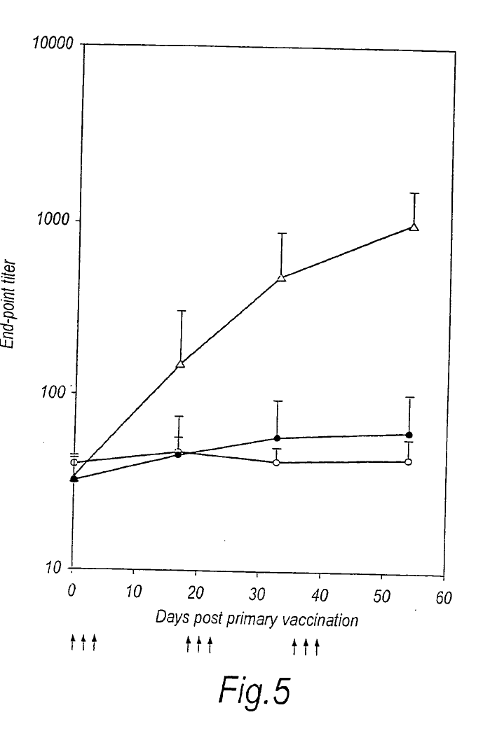

Two immunisations were performed. First, intra-peritoneal immunisation of Black C57 inbred mice (groups of 8) with formalin-inactivated cells (approx. 5×10⁹) expressing OppA-TTFC. FIG. 5 shows the serum IgG levels resulting from these immunisations and demonstrate the successful presentation and immunogencity of the OppA-TTFC chimera. To verify the immunogenicity of the double construct carrying OppA-TTFC and CotA-LTB we made both spores and vegetative cells and immunised (approx. 1×10⁹) by the IP route groups of 8 mice and followed the immune responses. Again high serum IgG levels were obtained by both routes.

e) Mucosal Immunity

To achieve mucosal immunity, we used oral dosing of groups of 8 inbred Black C57 mice. We show some examples in FIGS. 5-7.

First, oral administration of high concentrations of spores (1.7×10¹⁰) expressing OppA-TTFC (FIG. 5). As shown, we were able to achieve serum anti-TTFC specific IgG responses at essentially protective levels (usually reflected by titres higher than 10³). The only way a IgG response could be achieved is if a significant level of spore germination had occurred leading to immunity.

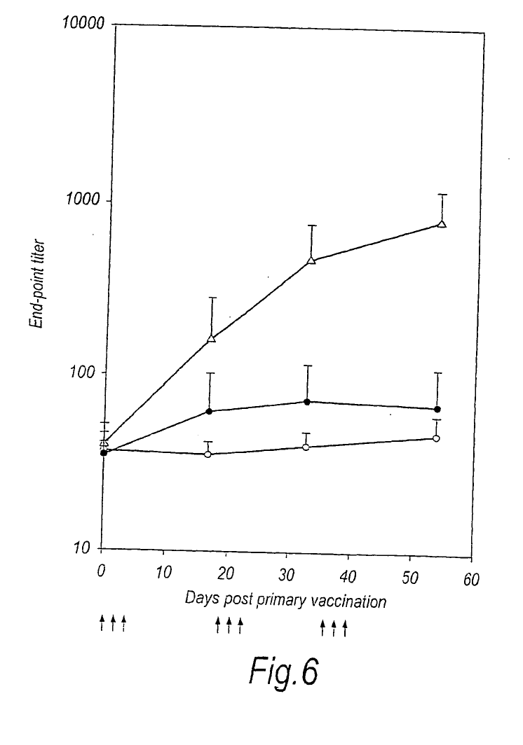

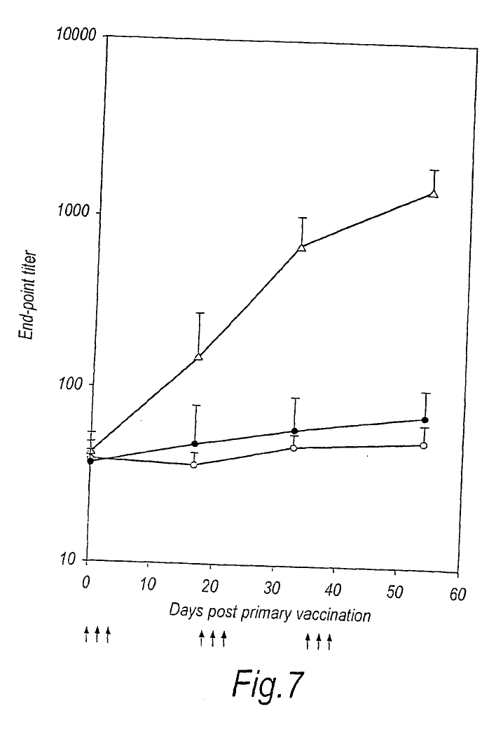

Second, oral dosing of mice (FIGS. 6 and 7) with spores carrying CotA-LTB and OppA-TTFC showed high serum IgG levels against both LTB and TTFC. This showed that multiple antigens could be displayed and used to generate immunity and opens the way for development as bivalent vaccine.

Other Applications

1) This strategy could be used to display any biologically active molecule. For example, an enzyme for an industrial application.

2) In accordance with the invention, spores could also be used with adjuvants to enhance the immune responses of the germinated cells. These might include, cholera toxin, chitosan or aprotonin.

Any combination of spore coat protein for spore expression together with any cell envelope protein for expression in the vegetative cell. That is, we are not restricted to CotA or OppA. Primary candidates for spore coat expression that we have identified are CotA, CotB, CotC, CotD, CotE and CotG.

Other Cell Surface Presentation Routes

We have used the OppA proteins as an example for presentation based primarily on ease of use and high levels of expression. Other cell envelope proteins could also be used including proteins involved in chemotaxis, solute-uptake etc. The only criteria is:
i) that the antigen can be fused to an exposed domain of the protein,
ii) the protein is present in the membrane at high levels

To use these types of protein would require an empirical approach systematically attempting presentation one at a time. Another approach is to use proteins that are associated with the peptidoglycan of the cell envelope, ie, the wall itself. In many Gram positives there are a group of “cell wall-anchored surface proteins” that are covalently attached to both the cytoplasmic membrane and peptidoglycan of the cell wall.

### EXAMPLE 2

Strains

SC2362 has been described elsewhere [1] and carries the rrnO-lacZ gene as well as the cat gene encoding resistance to chloramphenicol (5 mg/ml). rrnO is a vegetatively expressed gene encoding a rRNA. In this strain the 5′-region of rrnO carrying the promoter had been fused to the E. coli lacZ gene. PY79 is the prototrophic and isogenic ancestor of SC2362 and is Spo+[2]. DL169 (rrnO-lacZ gerD-cwlB D::neo) was created by transforming competent cells of strain TB1 (gerD-cwlB D::neo) with chromosomal DNA from SC2362 followed by selection for chloramphenicol resistance carried by the rrnO-lacZ cassette. TB1 has the gerD-cwlD region of the chromosome replaced with a neomycin-resistance gene and spores of this strain were found to have their rate of germination reduced to 0.0015% when compared to that of an isogenic wild type strain PY79 (E. Ricca; personal comm.).

Preparation of Spores and Vegetative Cells

Sporulation was made in DSM (Difco-sporulation media) media using the exhaustion method as described elsewhere [3]. Sporulating cultures were harvested 22 h after the initiation of sporulation. Purified suspensions of spores were made as described by Nicholson and Setlow [3] using lysozyme treatment to break any residual sporangial cells followed by successive washes in 1 M NaCl, 1 M KCl and then water (two-times). PMSF (10 mM) was included in washes to inhibit proteolysis. After the final suspension in water spores were treated at 68° C. for 1 h to kill any residual cells. Next, the spore suspension was titred immediately for cfu/ml before freezing aliquots at −20° C.

Vegetative B. subtilis cells were prepared by growth in LB containing 5% D-Glucose and 0.2% L-Glutamine until an OD600 nm corresponding to about 109 cfu/ml and used immediately. Growth under these conditions prevents inadvertent sporulation [4].

Analysis of Viable Bacteria in Faecal and Intestinal Tissues

Faecal counts were made by housing mice individually in cages with gridded floors to prevent coprophagia. Total faeces was collected at appropriate times and homogenised in PBS before plating serial dilutions on DSM (Difco sporulation medium; [5]) agar plates containing chloramphenicol (5 mg/ml) and Xgal (DSMCX) to select for SC2362 cells. Intestinal tissues were recovered from sacrificed mice and homogenised in PBS using glass beads (0.5 mm; 4×30 second bursts, 4° C.) before plating serial dilutions on DSMCX.

Simulated GIT Conditions

Bacteria were grown to a cell density corresponding to approx. 109 cells/ml in LB broth, harvested and suspended in simulated gastric juice (1 mg/ml pepsin {porcine stomach mucosa, Sigma}, pH 2.0) or small intestine fluid (0.2% bile salts {50% sodium cholate: 50% sodium deoxycholate; Sigma}, pH 7.4). The suspensions were incubated at 37° C., samples removed, serially diluted and plated for cfu/ml on LB agar plates.

Indirect ELISA for Detection of B-Galactosidase-Specific Serum Antibodies

Plates were coated with 50 ml/well of purified b-galactosidase (Sigma, 2 mg/ml in carbonate/bicarbonate buffer) and left at room temperature overnight. After blocking with 2% BSA in PBS for 1 h at 37° C. serum samples were applied using a 2-fold dilution series starting with a 1/40 dilution in ELISA diluent buffer (0.1 M Tris-HCl, pH 7.4; 3% (w/v) NaCl; 0.5% (w/v) BSA; 10% (v/v) sheep serum (Sigma); 0.1% (v/v) Triton-X-100; 0.05% (v/v) Tween-20). Every plate carried replicate wells of a negative control (a 1/40 diluted pre-immune serum), and a positive control (mouse anti-b-galactosidase (Sigma)). Plates were incubated for 2 h at 37° C. before addition of anti-mouse HRP conjugates (Sigma). Plates were incubated for a further 1 h at 37° C. then developed using the substrate TMB (3,3′, 5,5′-tetramethyl-benzidine; Sigma). Reactions were stopped using 2 M H₂SO₄. Dilution curves were drawn for each sample and endpoint titres calculated as the dilution producing the same optical density as the 1/40 dilution of a pooled preimmune serum. Statistical comparisons between groups were made by the Mann-Whitney U test. A P value of >0.05 was considered non-significant. To measure fecal IgA, a similar ELISA protocol was followed as described previously [6]. Samples were applied using a 2-fold serial dilution starting with undiluted faecal extract in PBS/2% BSA/0.05% Tween20. End-point titer was calculated as the dilution producing the same optical density as the undiluted pre-immune fecal extract. An end-point titer of 6.0 or greater was considered “positive”.

Extraction of Spore Coat Proteins and Vegetative Cell Lysates

Spore coat proteins were extracted from suspensions of spores of strain PY79 at high density (1×10¹⁰ spores/ml) using an SDS-DTT extraction buffer as described in detail elsewhere [3]. For vegetative cell lysates, strain PY79 was grown to an OD600 nm of 1.5 in LB medium and the cell suspension washed and then lysed by sonication followed by high speed centrifugation. Extracted proteins were assessed for integrity by SDS-PAGE and for concentration using the BioRad DC Protein Assay kit.

Immunisations

Groups of eight mice (female, BALB/C, 8 weeks) were dosed orally with suspensions (0.2 ml) of either spores or vegetative cells of either strain PY79, SC2362 or DL169. Mice were lightly anaesthetised with halothane. A naïve, non-immunised control group was included. Oral immunisations were administered by intra-gastric gavage on days 0, 1, 2, 20, 21, 22, 41, 42 and 43. Serum samples were collected on days −1, 18, 40 and 60, and fresh fecal pellets were collected on days −1, 18, 40 and 58. Faecal samples (0.1 g) were incubated overnight at 4° C. in 1 ml PBS/1% BSA/1 mM PMSF (phenylmethylsulphonyl fluoride, Sigma), then vortexed to disrupt all solid materials, and centrifuged at 13,000 rpm for 10 min. Sera and faecal extracts were stored at −20° C. until required.

Immunofluorescence Microscopy

B. subtilis strains (PY79 and SC2362) were grown to mid-log in LB medium. Samples were fixed in situ with 2.4% (w/v) paraformaldehyde, 0.04% glutaraldehyde and 0.03 M Na—PO₄ buffer pH 7.5 (final conc.) for 10 min at room temperature then 50 min on ice. The fixed bacteria were washed three times in PBS pH 7.4 at room temperature, then resuspended in GTE-lysozyme (50 mM glucose, 20 mM Tris-HCl pH 7.5, 10 mM EDTA, lysozyme 2 mg/ml). Aliquots (10 ml) were immediately applied on microscope cover glasses (BDH) that had been treated with 0.01% (w/v) poly-L-lysine (Sigma). After 4 min, the liquid was aspirated from the cover glass, which was then allowed to dry completely for 2 h at room temperature. The glass was washed 3 times in PBS pH 7.4, blocked for 15 min with 2% BSA in PBS at room temperature, then washed 9 more times. Samples were labelled with a 1:200 dilution of primary antibody (mouse anti-bgalactosidase) for 45 min at room temperature, washed 3 times, then incubated further with anti-mouse IgG-TRITC conjugate (Sigma) for 45 min at room temperature. After 3 washings, the cover glass was mounted onto a microscope slide and viewed under a Nikon Eclipse fluorescence microscope equipped with a BioRad Radiance 2100 laser scanning system. Images were taken using LaserSharp software and processed with the Confocal Assistant programme. Laser power was 30% for Green HeNe, scanning speed was 50 lps. Image size was 10×10 mm.

Results

Survival of B. subtilis in the Gastrointestinal Tract

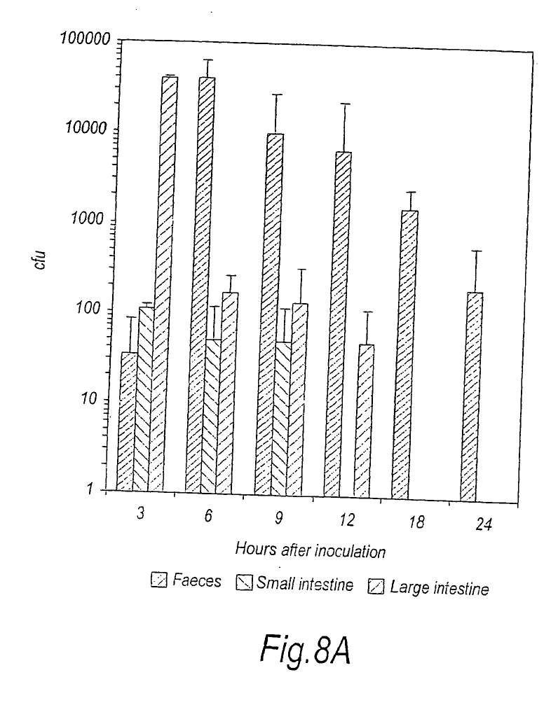

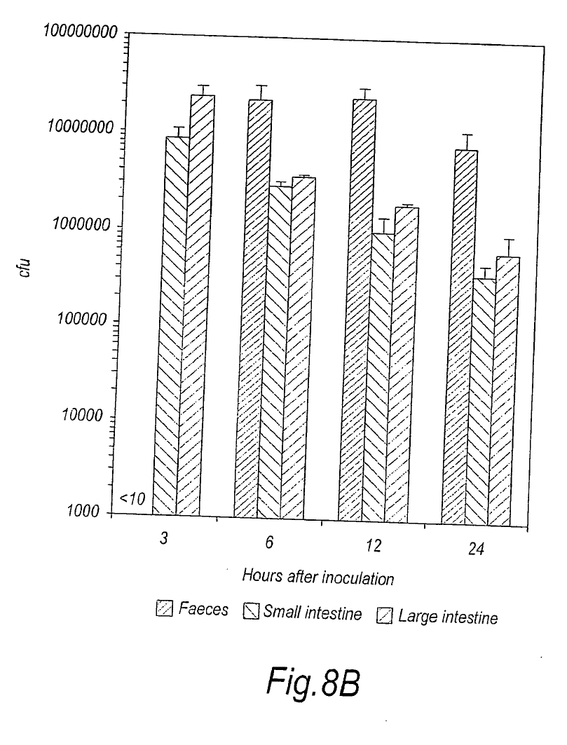

As a first step in developing spores for heterologous antigen delivery via the oral route we assessed the survival of B. subtilis vegetative cells and spores in the gastrointestinal tract of a murine model. To assess the robustness of the vegetative cell we inoculated two groups of inbred mice (Balb/c) each with a single dose of 2.4×10¹⁰ vegetative cells of strain SC2362 (rrnO-lacZ). One group of six mice was assessed for the number of viable counts of SC2362 that were present in the faeces collected from individually housed mice for the first 24 h after dosing (FIG. 8A). In this study the rrnO-lacZ marker enabled simplified identification and screening of viable colonies using the Lac+phenotype as well chloramphenicol resistance (encoded by the cat gene and carried by the rrnO-lacZ construct). Maximal counts of SC2362, corresponding to 0.00016% of the original dose, were found 6 h after dosing and these declined rapidly thereafter to insignificant levels by hour 24. The mean cumulative counts of SC2362 recovered in the faeces in the first 24 h corresponded to 0.00025% of the inoculating dose. With the second group of mice (12 animals) two animals were sacrificed at hours 3, 6, 9, 12, 18 and 24 and the small and large intestines were removed, homogenised and plated for counting of SC2362 viable units. As shown in FIG. 8A very low numbers of SC2362 were found in the small intestine with maximal counts being found at hour 3 (approx. 100). Higher counts (0.00016% of inoculating dose) were found in the large intestine at hour 3 but these counts declined thereafter.

To examine spore survival we performed a similar experiment to that described above but dosing orally with 2.1×10⁸ spores of strain SC2362 per mouse (FIG. 8B). Our assay technique differed from a previous study [7] in that the faeces was not heat-treated before plating and thus counts would include both spores as well as germinated spores (vegetative cells). Faecal counts from a group of six mice showed viable SC2362 present in the faeces at hour 6 in significant numbers with maximum levels at hour 12 (˜12% of inoculating dose). By hour 24 there were still considerable numbers of SC2362 counts (˜4%) present in the faeces. Counts in the small and large intestines showed similar kinetics as with dosing with vegetative bacteria (with maximal counts at hour 3) but with significantly higher levels of viable units. A group of five mice was also used as a naïve control of which one mouse was examined for faecal counts and the other four examined at appropriate time points for analysis of small and large intestinal counts. In each case no counts were recovered validating our assay technique.

Survival of B. subtilis in Simulated GIT Environments

We next asked what effect conditions within the GIT would have on the survival of both vegetative B. subtilis and intact spores using an in vitro assay. Based upon previous studies simulating conditions within the GIT [8-12] we recreated two environments, stomach and small intestine. Simulated conditions found in the stomach consisted of pepsin (1 mg/ml) at pH 2.0 in LB medium, and for the small intestine, 0.2% bile salts containing pancreatin (1 mg/ml) at pH 7.4 in LB. For assessing survival of spores though, LB was replaced with PBS since the nutrient rich LB medium might promote spore germination. Suspensions of vegetative B. subtilis cells or spores of strain SC2362 at approximately 108-109 cfu/ml were incubated in simulated stomach or small intestine conditions at 37° C. and survival determined by plating out and determination of cfu/ml.

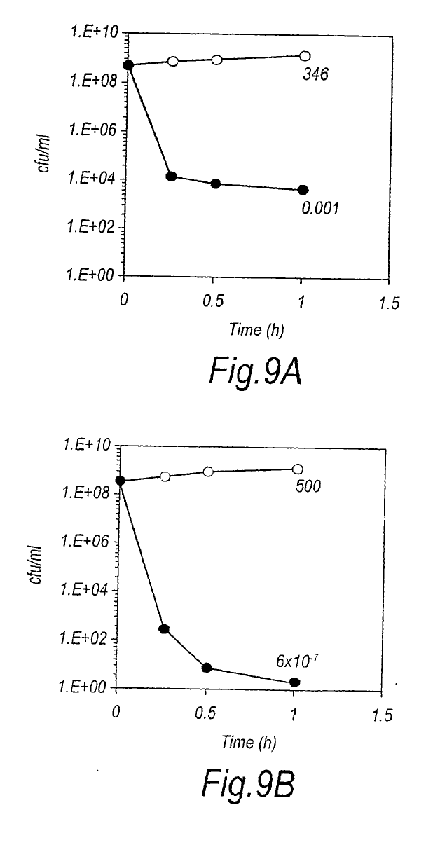

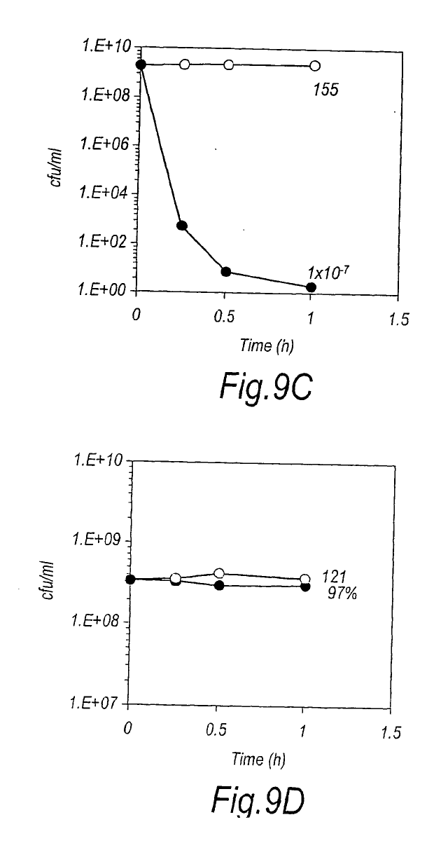

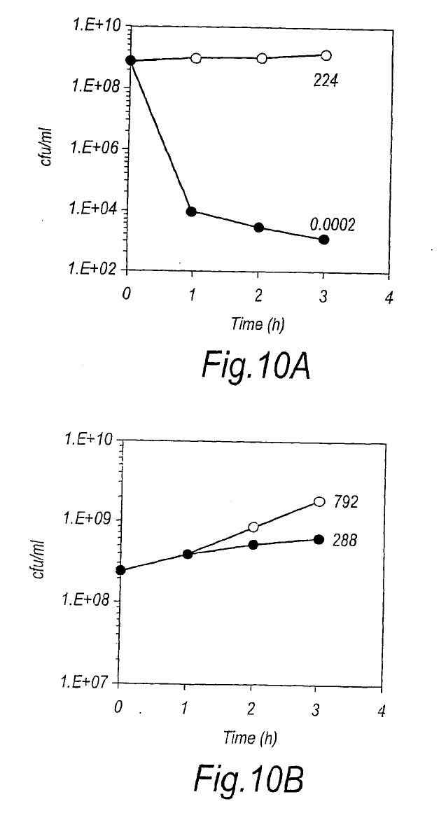

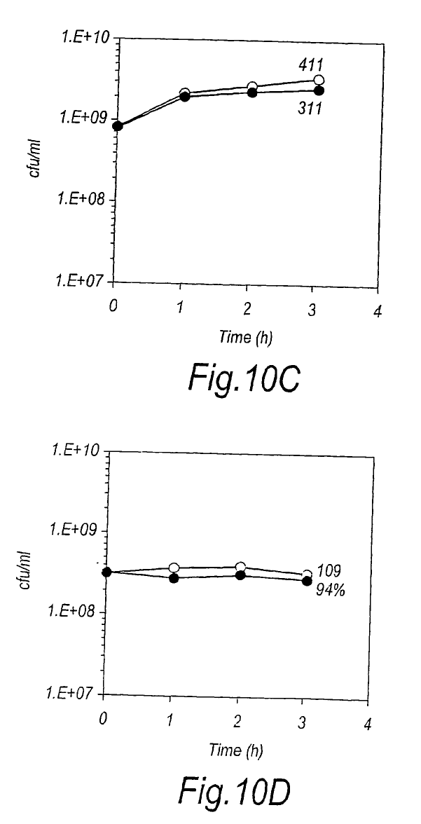

We also included two enteric bacterial species as controls, E. coli (strain BL21) and Citrobacter rodentium (ATCC 51459) the latter being a mouse pathogen that infects the small intestine [13]. As shown in FIG. 9 simulated gastric conditions resulted in a significant reduction in viability of vegetative cells of B. subtilis (FIG. 9A), E. coli (FIG. 9B) and C. rodentium (FIG. 9C) with almost complete loss of viability within 1 h. Spores were essentially unaffected though (FIG. 9D). Bile salts found in the small intestine, though, were found to have a significant effect on the viability of vegetative B. subtilis with only 0.0002% of the original inoculum surviving after the first hour (FIG. 10A). E. coli and C. rodentium however, were unaffected and could grow under these conditions with a moderate increase in cell numbers (FIGS. 10B and C). The effects on B. subtilis though, are primarily due to bile salts since in the absence of pancreatin cell viability was still substantially reduced to almost the same levels (data not shown). Finally, bile salts appeared to have no effect on intact spores (FIG. 10D).

Spore Germination in Simulated Intestinal Conditions

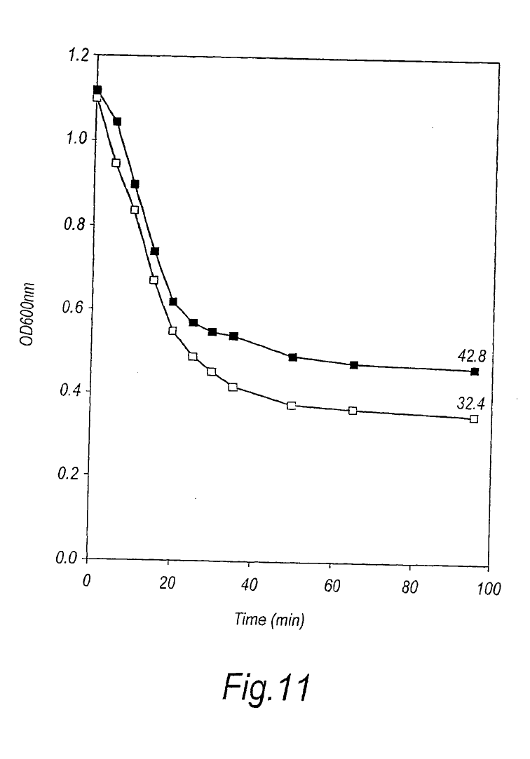

Upon entry into the duodenum spores have been shown to germinate [1, 7]. Since this region is rich in bile salts and our work has shown an effect of bile salts on cell viability we wondered what effect bile would have on spore germination. Using established procedures [3] we assessed germination in the presence or absence of 0.2% bile salts. A suspension of pure spores (wild type strain PY79) was incubated at 37° C. in the presence of specific germinants referred to as AGK (alanine-glucose-KCl). L-alanine was added at 10 mM (final conc.) to trigger spore germination and OD600 nm readings taken (FIG. 11). As spores germinate the OD declines as phase-bright spores lose their refractility and outgrow [14, 15]. Our results (repeated two times) showed that in the presence of AGK spore germination was extremely rapid with a 32.4% fall in OD600 nm in the first 90 minutes. In the presence of 0.2% bile salts though, spore germination was inhibited but not abolished with a 42.8% drop in OD600 nm over 90 minutes. This effect on spore germination has been observed previously [16] and is consistent with our more detailed findings here. In work not shown we have observed that spore germination is unaffected (ie, they do not germinate) in simulated stomach conditions.

Spores as an Antigen Delivery Vehicle

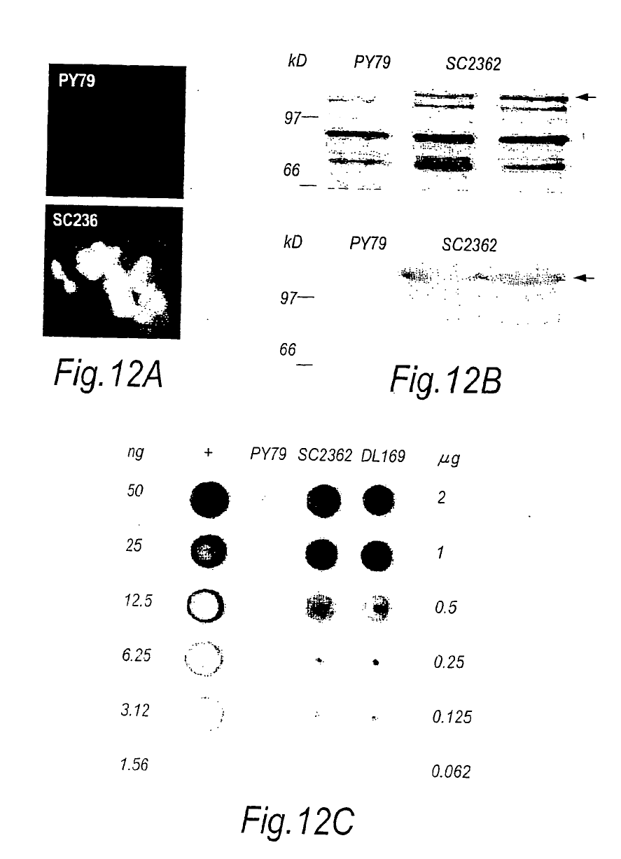

Our studies here demonstrate that spores are well equipped to survive transit across the stomach barrier. To address whether the spore could be used for heterologous antigen delivery we made use of the rrnO-lacZ gene carried in SC2362. rrnO-lacZ is itself a chimeric gene containing the strong, sA-recognised rrnO promoter fused to the lacZ gene of E. coli [1]. As a control, we constructed a germination mutant, DL169, which carried rrnO-lacZ together with a deletion (gerD-cwlB D::neo) in the gerD-cwlB region of the chromosome which is important for spore germination. Spores carrying the gerD-cwlB deletion are severely impaired (reduced to 0.0015% of wild type spores) in their ability to germinate (E. Ricca, pers. comm.). We verified that lacZ was expressed in vegetative cells of SC2362 by immunofluorescence as shown in FIG. 12A using a polyclonal sera against β-galactosidase. No detectable expression was found in the isogenic wild type strain PY79. SDS-PAGE analysis of fractionated whole cell extracts of SC2362 and DL169 cells (FIG. 12B) revealed a predominant band at 117 kD corresponding to the size of β-galactosidase. Western blotting with a polyclonal anti-β-galactosidase antibody confirmed this and showed a number of high mwt. breakdown products but otherwise no obvious degradation.

A quantitative determination of the amount of β-galactosidase expressed in SC2362 cells expressing rrnO-lacZ was obtained by dot blot experiments using serial dilutions of purified β-galactosidase (Sigma) and of whole cell extracts of B. subtilis strains PY79, SC2362 and DL169 (FIG. 5C). Proteins were reacted with an anti-β-galactosidase polyclonal antibody, then with alkaline phosphatase-conjugated secondary antibodies and colour developed by the BCIP/NBT or ECL system (Bio-Rad). A densitometric analysis indicated that no β-galactosidase was detectable in PY79 cells. In SC2362 and DL169 cell extracts the amount of β-galactosidase equated to 3.14% (31.4 ng/mg) of total extracted protein for SC2362 and 2.4% (24 ng/mg) of total extracted protein for DL169 (average of 0.43 mg). The high levels of β-galactosidase produced in these strains were confirmed by the SDS-PAGE analysis (FIG. 12B) and demonstrate the efficacy of the rrnO promoter for heterologous gene expression.

Serum Anti-β-Galactosidase Responses Following Oral Delivery of Spores Carrying rrnO-lacZ

Groups of seven inbred mice were dosed orally with spores or vegetative cells of SC2362, DL169 or PY79. We used a dosing regime previously optimised for oral immunisations [6] and each immunising dose contained either 2×10¹⁰ spores or 3×10¹⁰ vegetative cells. From our densitometric analysis (see the section above entitled “Spores as an antigen delivery vehicle”) we could define one dose of SC2362 or DL169 vegetative cells as containing approximately 0.43 mg of β-galactosidase.

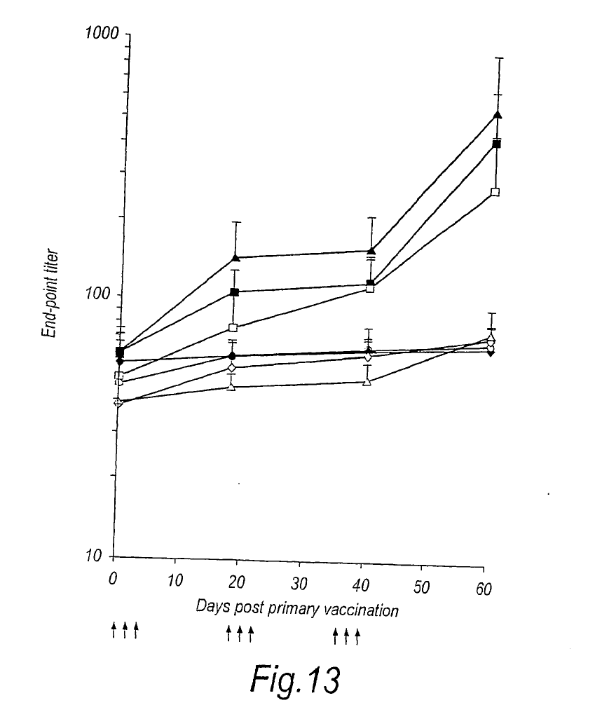

Serum samples were analysed by ELISA for anti-β-galactosidase IgG (FIG. 13) and as a control we also included a group of seven non-immunised mice for sampling. As shown in FIG. 13 oral immunisation of mice with SC2362 (rrnO-lacZ) spores gave end point titres significantly above (P<0.05) those of mice dosed with nonrecombinant spores (PY79) or the control naïve group from day 40 onwards. DL169 spores, though, failed to produce seroconversion in immunised mice and anti-α-galactosidase titres did not significantly (P>0.05) differ from those of mice dosed with non-recombinant spores (PY79) or the control naïve group. This then shows clearly that a proportion of SC2362 spores must have germinated following oral delivery leading to subsequent expression of rrnO-lacZ. Failure to generate these responses following delivery of DL169 spores proves that spore germination is essential for generating these humoral responses. At this stage, we are not concerned with the levels of antibody responses but rather proof that spore germination can be used for antigen delivery. Immunisations using vegetative cells of each strain were incorporated as controls and we were somewhat surprised to detect anti-β-galactosidase IgG responses in mice dosed with SC2362 or DL169 cells. The levels of responses were similar to those obtained from dosing with SC2362 spores (FIG. 13) and the similarity in responses may imply a threshold level has been reached when using this dosing regime and with β-galactosidase as the immunogen.

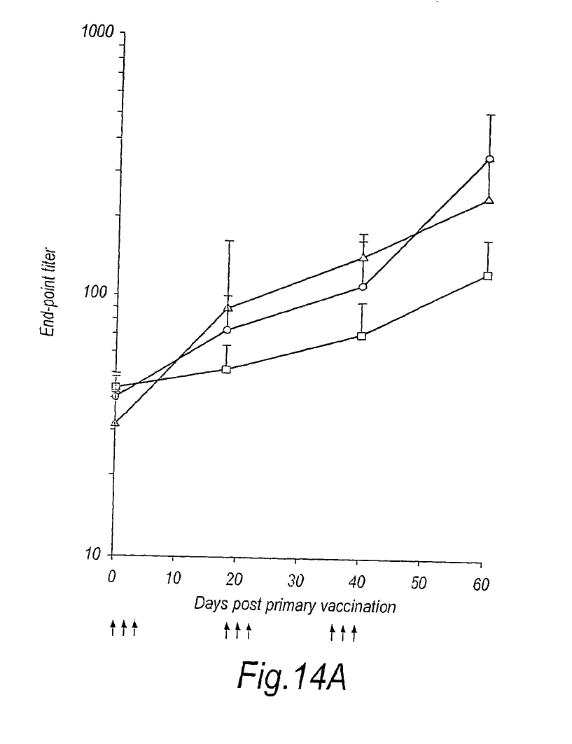

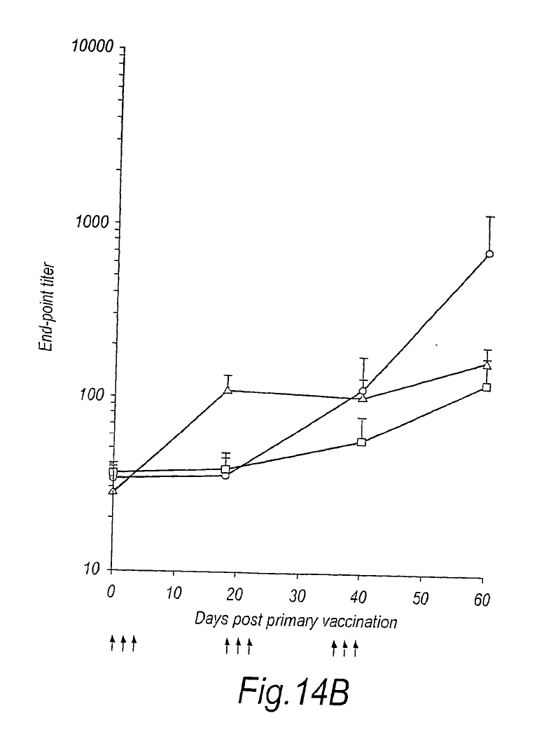

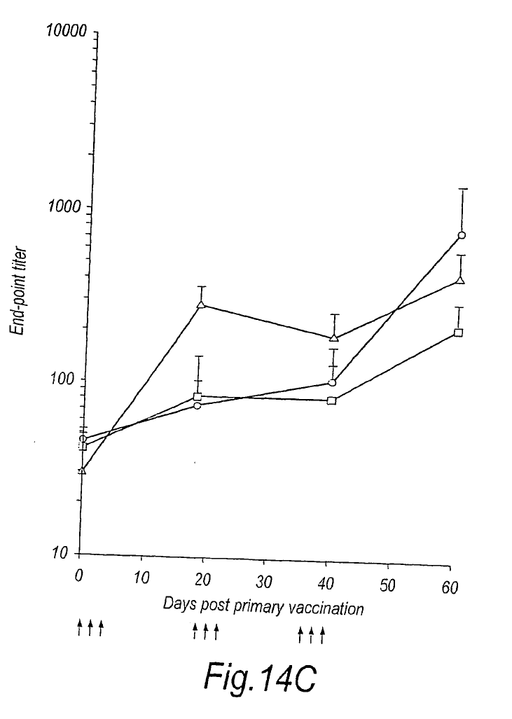

Sera from mice immunised with SC2362 spores (FIG. 14A), SC2362 vegetative cells (FIG. 14B) and DL169 vegetative cells (FIG. 14C) was also examined for the presence of β-galactosidase-specific IgG1, IgG2a and IgG2b subclasses. Immunisation with vegetative cells of either SC2362 or DL169 showed IgG2a to the first detectable subclass at day 20 followed by a gradual increase in IgG1. Dosing with SC2362 spores showed an early increase in both IgG1 and IgG2a. In all three cases the levels of IgG2b increased more slowly.

Mucosal Anti-β-Galactosidase Responses Following Oral Delivery of Spores Carrying rrnO-lacZ

Out of eight mice, only one in the group receiving SC2362 spores gave a positive titer of 16.8 on day 58. The level of anti-b-galactosidase-specific faecal IgA in the group immunised with vegetative cells of the same strain was higher with 3, 1 and 4 out of 8 mice having positive responses on days 18, 40 and 58 respectively (data not shown). Finally, the group immunised with DL169 spores go no positive responses and with DL169 vegetative cells only one positive response on day 18. No positive titers were found with other groups.

Discussion

The aim of Example 2 is to evaluate B. subtilis spores as an oral vaccine delivery system. Our rationale was based on several attributes that would make spores a particularly promising vaccine vehicle. First, their current use as a probiotic for human and animal use. Second, they are non-pathogenic microorganisms normally found in the soil. Third, as robust and dormant life forms they would be suitable for long term storage in the dessicated (spore) form. Fourth, as a model unicellular differentiating (sporeforming) organism genetic analysis in this organism is second to none and supported by excellent cloning technology. Finally, this organism when administered orally in the spore state can germinate and undergo limited rounds of replication and cell growth in the small intestine before being excreted. Based on the ability of spores to germinate in the GIT we have investigated the germinating spore as the mechanism for heterologous antigen delivery. The logic and novelty of our approach is that the spore might be able to survive transit across the stomach after which it would germinate and then in the vegetative phase express the heterologous antigen.

Before evaluating specific humoral responses we evaluated the survival of spores as well as vegetative cells in the GIT tract. Using an in vivo analysis in mice we have found that spores are essentially unaffected when given orally with most being excreted after 24 h. By contrast, vegetative B. subtilis cells have a very low survival in the mouse GIT. As an approximation we estimate less than 0.0005% of vegetative cells survive transit through the GIT. The stomach would likely be the first and most severe barrier to vegetative B. subtilis and this is supported by the extremely low levels of viable counts recovered in the small intestine. For those bacteria that do survive they appear to have transited the stomach within the first three hours after dosing and were present in the faeces by the 6^{th} hour.

We supported these observations by using an in vitro assay where spore or vegetative cell survival was assessed in simulated conditions mimicking the stomach or small intestine. These results showed that for vegetative B. subtilis there is a limited chance for long-term survival in the stomach or small intestine. The simulated stomach environment appeared to present a hostile environment not only to B. subtilis but also to other enteric bacteria such as E. coli and C. rodentium. For B. subtilis this is shown from our direct counting experiments of small intestine tissues where clearly some percentage of cells have survived transit across the stomach. Presumably, in vivo, this is due to the effects of clumping and aggregation, transit time and the composition the stomach. For B. subtilis a second barrier comprised of the effects of bile salts is presented upon exit from the stomach which would ensure almost no survival and is supported by our in vivo experiments described above where we estimate less than 0.0005% of vegetative bacteria can survive transit through the GIT. Spores, as might be expected, can survive such harsh conditions with no deleterious effect.

The effect of bile salts on B. subtilis demonstrate the inability of this organism to survive, long term, in the GIT, in contrast to enteric microbes. The effect of bile salts on spores and vegetative cells was interesting. While bactericidal on vegetative cells their effect on spores was a modest inhibition of germination. Thus, spores exiting the stomach would initially be inhibited from germinating but those that did germinate would be killed. These opposed effects though, would be modulated by the precise composition of the intestinal lumen as well as the distance passed after exit from the stomach.

We have used β-galactosidase as the model antigen to evaluate our vaccine hypothesis since this protein has been used successfully to evaluate new vaccine delivery systems [17, 18]. Our analysis of systemic anti-β-galactosidase IgG responses to orally delivered spores proves that spores can indeed germinate and synthesise sufficient immunogen to generate the observed seroconversion. This would validate our hypothesis and demonstrate the potential of spores as vaccine vehicles. We have shown that a proportion of spores can germinate in the small intestine and presumably these enter the GALT at this region. Alternatively, intact spores may transit the mucosa and germinate within the GALT (eg, in the Peyer's Patches). The small size (1-1.2 microns) of the spore particle make this a distinct possibility since they are small enough to be taken up by M cells.

Generation of secretory IgA responses are obviously beneficial for any mucosal vaccine and local responses to β-galactosidase were low in this pilot study although some mice did show responses. Most probably this reflects the relatively low immunogenicity of β-galactosidase but might also reflect the dosing regime. Interestingly, we obtained similar responses when vegetative cells were used for antigen delivery. These were used as controls yet despite the fact that we predicted almost 100% cell death in the stomach sufficient β-galactosidase could be delivered to generate the observed anti-β-galactosidase IgG titres. We can estimate the oral dose of antigen as approximately 0.43 mg which was given nine times. Presumably, the responses we observe come from intact vegetative cells that have transited the stomach and entered the small intestine which is responsible for generating humoral responses of orally administered antigens. We can not say from this study whether killed B. subtilis cells can generate the observed humoral responses but we would predict that it does not matter whether the cell is alive or dead. At first glance it might not seem obvious why the spore state is advantageous since both forms can induce local and systemic responses. The spore state though, offers the benefits of long term storage (perhaps in terms of decades) in the dessicated state at ambient temperature.

### REFERENCES

[1] Casula, G. and S. M. Cutting. Bacillus Probiotics: Spore Germination in the Gastrointestinal Tract. Applied and Environmental Microbiology 2002; 68:2344-2352.
[2] Youngman, P., J. Perkins, and R. Losick. Construction of a cloning site near one end of Tn917 into which foreign DNA may be inserted without affecting transposition in Bacillus subtilis or expression of the transposon-borne erm gene. Plasmid 1984; 12:1-9.
[3] Nicholson, W. L. and P. Setlow, Sporulation, germination and outgrowth., in Molecular Biological Methods for Bacillus., C. R. Harwood. and S. M. Cutting., Editors. 1990, John Wiley & Sons Ltd.: Chichester, UK. p. 391-450.
[4] Schaeffer, P., J. Millet, and J. Aubert. Catabolic repression of bacterial sporulation. Proceedings of the National Academy of Sciences USA 1965; 54:704-711.
[5] Cutting, S. M. and P. B. Vander-Horn, Genetic Analysis, in Molecular Biological Methods for Bacillus, C. R. Harwood and S. M. Cutting, Editors. 1990, John Wiley & Sons Ltd.: Chichester, England. p. 27-74.
[6] Robinson, K., et al. Oral vaccination of mice against tetanus with recombinant Lactococcus lactis. Nature Biotechnology 1997; 15:653-657.
[7] Hoa, T. T., et al. The Fate and Dissemination of B. subtilis spores in a murine model. Applied and Environmental Microbiology 2001; 67:3819-3823.
[8] Almirall, M. and E. Esteve-Garcia. The stability of a b-glucanase preparation from Trichoderma longibrachiatum and its effect in a barley based diet fed to broiler chicks. Animal Feed Science & Technology 1995; 54:149-158.
[9] Araujo, A. H., et al. In vitro digestibility of globulins from cowpea (Vigna unguiculata) and xerophitic algaroba (Prosopsis juliflora) seeds by mammalian digestive proteinases: a comparative study. Food Chemistry 2002; 78:143-147.
[10] Aungst, B. J. and S. Phang. Metabolism of a neurotensin (8-13) analog by intestinal and nasal enzymes, and approaches to stabilize this peptide at these absorption sites. International Journal of Pharmaceutics 1994; 117:95-100.
[11] Freund, O., et al. In vitro and in vivo stability of new multilamellar vesicles. Life Sciences 2000; 67:411-419.
[12] Lebet, V., E. Arrigoni, and R. Amado. Digestion procedure using mammalian enzymes to obtain substrates for in vitro fermentation studies. Lebensm. Wiss. u. Technol. 1998; 31:509-515.
[13] Higgins, L. M., et al. Citrobacter rodentium infection in mice elicits a mucosal Th1 cytokine response and lesions similar to those in murine inflammatory bowel disease. Infect. Immun. 1999; 67:3031-3039.
[14] Higgins, L. M., et al. Citrobacter rodentium infection in mice elicits a mucosal Th1 cytokine response and lesions similar to those in murine inflammatory bowel disease. Infect. Immun. 1999; 67:3031-3039.
[15] James, W. and J. Mandelstam. spoVIC, a new sporulation locus in Bacillus subtilis affecting spore coats, germination and the rate of sporulation. Journal of General Microbiology 1985; 131:2409-2419.
[16] Spinosa, M. R., et al. On the fate of ingested Bacillus spores. Research in Microbiology 2000; 151:361-368.
[17] Barletta, R. G., et al. Recombinant BCG as a candidate oral vaccine vector. Research in Microbiology 1990; 141:931-939.
[18] Gheradi, M. M. and M. Esteban. Mucosal and systemic immune responses induced after oral delivery of vaccinia virus recombinants. Vaccine 1999; 17:1074-1083.

1-31. (canceled)

32. A Bacillus spore which is genetically modified with genetic code comprising at least one genetic construct encoding a therapeutically active compound and a targeting sequence or a vegetative cell protein for use in oral administration for therapeutic treatment.

33. A spore as claimed in claim 32, wherein the therapeutically active compound is an antigen or a medicament or a precursor to an antigen or a medicament.

34. A spore as claimed in claim 32, wherein the gene construct is a chimeric gene.

35. A spore as claimed in claim 33, wherein the gene construct is a chimeric gene.

36. A spore as claimed in claim 32, wherein the genetic modification is accomplished by transformation of a mother cell using a vector containing the gene construct and then inducing the mother cell to produce the spores.

37. A spore as claimed in claim 32, wherein the gene construct is under the control of one or more of, each or independently, an inducible promoter, a promoter or a strong promoter or modified promoter.

38. A spore as claimed in claim 37, wherein the gene construct is under the control of one or more of, each or independently, an inducible promoter, a promoter or a strong promoter or modified promoter.

39. A spore as claimed in claim 37, wherein the gene construct has an enhancer element or an upstream activator sequence associated with it.

40. A spore as claimed in claim 32, wherein the construct comprises an inducible expression system.

41. A spore as claimed in claim 37, wherein the construct comprises an inducible expression system.

42. A spore as claimed in claim 32, wherein the spore germinates in the duodenum and/or the jejunum of an intestinal tract of a human or animal body.

43. A spore as claimed in claim 32, wherein the therapeutically active compound is an antigen which, in use, is adapted to elicit an immune response.

44. A spore as claimed in claim 43, wherein the antigen is at least a fragment of tetanus toxin fragment C or labile toxin B sub unit.

45. A spore as claimed in claim 37, wherein the protein is a protein that is expressed in the cell barrier.

46. A spore as claimed in claim 45, wherein the protein is a protein that is expressed in the cell barrier.

47. A spore as claimed in claim 37, wherein the protein is expressed all the time in a vegetative cell.

48. A spore as claimed in claim 47, wherein the protein is expressed all the time in a vegetative cell.

49. A spore as claimed in claim 47, wherein the protein is OppA or rrnO.

50. A spore as claimed in claim 32, wherein the protein is expressed intermittently in a vegetative cell.

51. A spore as claimed in claim 46, wherein the protein is expressed intermittently in a vegetative cell.

52. A spore as claimed in claim 32, wherein the protein is a soluble cytoplasmic vegetative cell protein.

53. A spore as claimed in claim 44, wherein the protein is a soluble cytoplasmic vegetative cell protein.

54. A spore as claimed in claim 52, wherein the protein is rrnO.

55. A spore as claimed in claim 52, wherein the genetic construct of the soluble cytoplasmic protein wholly or partially comprises a signal sequence.

56. A spore as claimed in claim 54, wherein the genetic construct of the soluble cytoplasmic protein wholly or partially comprises a signal sequence.

57. A spore as claimed in claim 32, wherein the signal sequence is adapted to target the therapeutically active compound to a specific part of the vegetative cell.

58. A spore as claimed in claim 44, wherein the signal sequence is adapted to target the therapeutically active compound to a specific part of the vegetative cell.

59. A spore as claimed in claim 57, wherein the signal sequence directs the therapeutically active compound for secretion (preferably active secretion, more preferably Type I, Type II or Type III secretion), or for post-translational processing by a vegetative cell (preferably glycosylation).

60. A spore as claimed in claim 32, wherein the therapeutically active compound is an antigen precursor which is one or more enzymes capable of transforming a biological precursors, such that upon germination said one or more enzymes are expressed and synthesise one or more antigens by transformation of a said biological precursor.

61. A spore as claimed in claim 59, wherein the therapeutically active compound is an antigen precursor which is one or more enzymes capable of transforming a biological precursors, such that upon germination said one or more enzymes are expressed and synthesise one or more antigens by transformation of a said biological precursor.

62. A spore as claimed in claim 60, wherein the biological precursor is a hormone, a steroid hormone, a painkiller or a pro-drug.

63. A spore as claimed in claim 32, wherein the therapeutically active compound is a medicament which is a protein, a vaccine or an endorphin.

64. A spore as claimed in claim 59, wherein the therapeutically active compound is a medicament which is a protein, a vaccine or an endorphin.

65. A spore as defined in claim 32, wherein it is for use in treatment of a medical condition, preferably the medical condition is inflammation, pain, a hormonal imbalance and/or an intestinal disorder.

66. A spore as defined in claim 64, wherein it is for use in treatment of a medical condition, preferably the medical condition is inflammation, pain, a hormonal imbalance and/or an intestinal disorder.

67. A composition comprising at least two different spores as defined in claim 32, wherien said at least two different spores express at least two different therapeutically active compounds.

68. A composition as defined in claim 67, wherein the composition further comprises a pharmaceutically acceptable excipient or carrier.

69. A composition comprising a spore as defined in claim 32 in association with a pharmaceutically acceptable excipient or carrier.

70. A composition comprising a spore as defined in claim 65 in association with a pharmaceutically acceptable excipient or carrier.

71. A composition as defined in claim 67 for use in treatment of a medical condition, preferably the medical condition is inflammation, pain, a hormonal imbalance and/or an intestinal disorder.

72. A composition as defined in claim 68 for use in treatment of a medical condition, preferably the medical condition is inflammation, pain, a hormonal imbalance and/or an intestinal disorder.

73. A composition as defined in claim 69 for use in treatment of a medical condition, preferably the medical condition is inflammation, pain, a hormonal imbalance and/or an intestinal disorder.

74. Use of a spore as defined in claim 32 in the manufacture of a medicament for use in the treatment of a medical condition, preferably the medical condition is inflammation, pain, a hormonal imbalance and/or an intestinal disorder.

75. A method of medical treatment, which method comprises the steps of
a) administering a spore as defined in claim 32 to a human or animal in need of medical treatment;
b) said spore germinating into a vegetative cell in the intestinal tract;
c) said vegetative cell expressing a therapeutically active compound for use in the medical treatment.

76. A method of medical treatment, which method comprises the steps of
d) administering a spore as defined in claim 65 to a human or animal in need of medical treatment;
e) said spore germinating into a vegetative cell in the intestinal tract;
f) said vegetative cell expressing a therapeutically active compound for use in the medical treatment.

77. A method as claimed in claim 75, wherein the spore is administered orally, intra-nasally or rectally.

78. A method as claimed in claim 76, wherein the spore is administered orally, intra-nasally or rectally.
